(19) United States
(12) Patent Application Publication
(10) Pub. No.: US 2012/0328670 A1
(43) Pub. Date: Dec. 27, 2012

(54) Targeted Delivery of Retinoid Compounds to the Sebaceous Glands

(75) Inventors: John E. Donello, Dana Point, CA (US); Rong Yang, Mission Viejo, CA (US)

(73) Assignee: ALLERGAN, INC., Irvine, CA (US)

(21) Appl. No.: 13/486,137

(22) Filed: Jun. 1, 2012

Related U.S. Application Data
(60) Provisional application No. 61/493,341, filed on Jun. 3, 2011.

(51) Int. Cl. A61K 9/14; A61P 17/10; A61P 17/00; A61K 31/4436

(52) U.S. Cl. 424/400; 514/337

(57) ABSTRACT

Disclosed herein are topical dermal compositions comprising particles, wherein the particles comprise

a) a biodegradable polymer, and

b) a retinoid selected from the group consisting of:

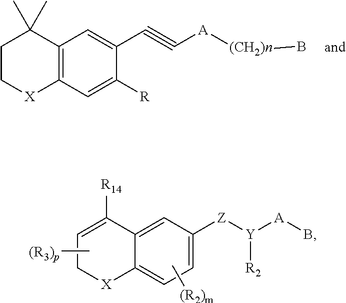

or a pharmaceutically acceptable salt thereof, wherein the particles have an average diameter between 0.1 μm and 10 μm, and wherein the variables are as defined in the specification. The compositions are useful for treating a condition associated with excess sebum production.

### CROSS REFERENCE TO RELATED APPLICATION

This Application claims the benefit of U.S. Provisional Application Ser. No. 61/493,341 filed on Jun. 3, 2011, which is incorporated herein by reference in its entirety.

### BACKGROUND

Human skin is composed of three primary layers: the stratum corneum, the epidermis, and the dermis. The outer layer is the stratum corneum. Its primary function is to serve as a barrier to the external environment. Lipids are secreted to the surface of the stratum corneum, where they decrease the stratum corneum's water permeability. Sebum typically constitutes 95% of these lipids. Abramovits et al., Dermatologic Clinics, 18:4 (2000). In addition to maintaining the epidermal permeability barrier, sebum transports anti-oxidants to the surface of the skin and protects against microbial colonization.

Sebum is produced in the sebaceous glands. These glands are present over most of the surface of the body. The highest concentration of these glands occurs on the scalp, the forehead, and the face. Despite the important physiological role that sebum plays, many individuals experience excess sebum production, especially in the facial area. An increased rate of sebum excretion is termed seborrhoea.

Seborrhoeic dermatitis is also associated with seborrhea. The condition is characterized by the appearance of red, flaking, greasy areas of skin, most commonly on the scalp, nasolabial folds, ears, eyebrows and chest. In the clinical literature seborrhoeic dermatitis may be also referred to as “sebopsoriasis,” “seborrhoeic eczema,” “dandruff,” and “pityriasis capitis.” Yeast infections are a causative factor in seborrhoeic dermatitis. The yeast thrives on sebum and leaves high concentrations of unsaturated fatty acids on the skin, thereby irritating it.

Acne vulgaris is associated with clinical seborrhea and there is a direct relationship between the sebum excretion rate and the severity of acne vulgaris. Although sebum production increases during adolescence (particularly in boys, because of androgen stimulation), increased sebum alone does not cause acne. Bacteria, most importantly P. acnes, feed on sebum and as a result are present in increased numbers in persons who have acne. Much of the inflammation associated with acne arises from the action of enzymes produced by the bacteria.

Acne vulgaris is characterized by areas of skin with seborrhea (scaly red skin), comedones (blackheads and whiteheads), papules (pinheads), pustules (pimples), nodules (large papules), and in more severe cases, scarring. It mostly affects skin with the densest population of sebaceous follicles, such as the face, upper chest, and back. There are four key pathogenic factors of acne:

Follicular hyperkeratinization

Propionibacterium acnes (P. acnes)

Inflammation

Excessive sebum production (seborrhea)

Acne is still a very underserved market with treatment options that are only marginally effective. Only one product, oral Accutane® (isotretinoin) that reduces sebum production has been highly effective, but at the expense of a black box warning with significant side effects including teratogenicity that require extensive patient monitoring. Accutane® is indicated only for acne which is severe and recalcitrant to other treatment. There is a need in the art, therefore, for additional agents capable of reducing sebum production and treating the conditions associated with it.

Topical therapy is often preferred over oral therapy because of the reduced risk for adverse systemic effects. The most common topical drugs for acne can be divided into the following categories:

Retinoids (ie., tazarotene, tretinoin, adapalene)

Antibiotics (ie., clindamycin)

Benzoyl peroxide (BPO)

Others (i.e., dapsone, azelaic acid)

While many topical therapies are available, none of them address all four factors and most specialize in a few of these factors. Currently, no topical therapies in the market address excessive sebum production. Sebum is produced by the sebaceous gland, which is an appendage of the hair follicle, so it makes sense to target the sebaceous gland for more effective therapy. Since P. acnes depends on sebum to live, reduction of sebum is also thought to indirectly reduce P. acnes.

Topical retinoids primarily act by normalizing infundibular hyperkeratinization and reducing inflammation, hence topical retinoids remain a mainstay for treatment of mild-to-moderate acne. The current topical retinoid formulations do not inhibit sebum production and their use is often limited by local tolerability (i.e., skin irritation).

Tazarotene is a RAR (retinoic acid receptor) β/γ-selective RA (retinoic acid) that is approved for the topical treatment of acne and psoriasis. The current topical formulations of tazarotene (cream, gel) do not inhibit sebum production, presumably because these formulations do not enable high selective drug exposure to the sebaceous gland. Therefore, there is a need for a novel approach to provide targeted delivery of tazarotene to the sebaceous gland The present invention is directed to dermal topical compositions comprising encapsulated retinoid compounds (such as tazarotene).

R. Toll et al., J Invest Dermatol 123:168-176, 2004, refer to penetration profile of microspheres in follicular targeting of terminal hair follicles.

Christian Wischke et al., International Journal of Pharmaceutics 364 (2008) 298-327 refers to principles of encapsulating hydrophobic drugs in PLA/PLGA microparticles.

Annika Vogt et al., J Investig Dermatol Symp Proc 10:252-255, 2005, refers to follicular targeting—a promising tool in selective dermatotherapy.

Alain Rolland et al., Pharmaceutical Research, Vol 10, No. 12, 1993 refer to site-specific drug delivery to pilosebaceous structures using polymeric microspheres.

Gisele A. Castro et al., International Journal of Pharmaceutics 381 (2009) 77-83, refer to formation of ion pairing as an alternative to improve encapsulation and stability, and to reduce skin irritation of retinoic acid loaded in solid lipid nanoparticles.

### SUMMARY OF THE INVENTION

The inventors have found that certain retinoid compounds, when formulated as described herein, penetrate the hair follicle to the depth of the sebaceous gland, thereby permitting the retinoid compounds to act directly on the gland. Accordingly, in one embodiment, the formulations permit the retinoid compounds to exert stronger and more selective effects on the gland, which enables inhibition of sebum production, thereby decreasing overall disease symptomology. These formulations may also simultaneously reduce drug associated skin side effects (such as irritation) and risks associated with systemic drug exposure. In another embodiment, the formulations enable slow release of the compound that expose the target tissue to a more constant level of drug that reduce the total drug needed to treat the condition, which may also improve efficacy and tolerability.

In accordance with these embodiments, disclosed herein is a dermal topical composition comprising particles, wherein the particles comprise

a) a biodegradable polymer, and

b) a compound of the formula:

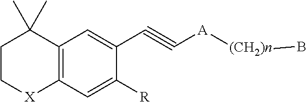

wherein:

X is S, O, or —N(R¹)— where R¹ is hydrogen or lower alkyl;

R is hydrogen or lower alkyl;

A is pyridinyl, thienyl, furyl, pyridazinyl, pyrimidinyl or pyrazinyl;

n is 0-2;

B is selected from the group consisting of: H, —COOH or a pharmaceutically acceptable salt, ester or amide of said —COOH group, —CH₂OH or an ether or ester derivative of said —CH₂OH group , —CHO or an acetal derivative of said —CHO group, and —COR² or a ketal derivative of said —COR² group, wherein R² is —(CH₂)ₘCH₃ wherein m is 0-4; and

wherein the particles have an average diameter between about 0.1 μm and about 10 μm.

In another embodiment, the present invention provides a dermal topical composition comprising particles, wherein the particles comprise

a) a biodegradable polymer, and

b) a compound of the formula:

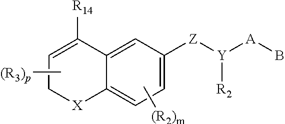

or a pharmaceutically acceptable salt thereof, wherein X is S, O, NR′ where R′ is H or alkyl of 1 to 6 carbons, or

X is [C(R₁)₂]ₙ where R₁ is independently H or alkyl of 1 to 6 carbons, and n is an integer between, and including, 0 and 2, and;

R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;

R₃ is hydrogen, lower alkyl of 1 to 6 carbons or F, and;

m is an integer having the value of 0-3, and;

p is an integer having the value of 0-3, and;

Z is —C≡C—, —N═N—, —N═CR₁—, —CR₁═N, —(CR₁═CR₁)ₙ′— where n′ is an integer having the value 0-5, —CO—NR₁—, —CS—NR₁—, —NR₁—CO, —NR₁—CS, —COO—, —OCO—; —CSO—; —OCS—;

Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′} group and B;

A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;

B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and

R₁₄ iS (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and

R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons; and

wherein the particles have an average diameter between about 0.1 μm and about 10 μm.

The present invention also provides a method for treating a condition associated with excess sebum production, comprising topically applying to the skin of a patient in need thereof any of the aforementioned topical dermal compositions.

### BRIEF DESCRIPTION OF THE DRAWINGS

FIG. 1 shows the ability of tazarotene microspheres having an average diameter of about 4.2 μm to reduce sebaceous gland size in the hamster flank organ model. Six animals were in this treatment group. The right side flank organ is treated with tazarotene microsphere containing 0.03% tazarotene. The untreated left side is used as control. Animals were treated 5 days/week for 26 days. Slices stained with hematoxylin and eosin show abundant sebaceous glands in the flank organs from the control side and much reduced sebaceous glands in the flank organs treated with tazarotene microsphere. Arrows point to atrophied sebaceous glands in the treated side.

FIG. 2 shows combined results from several studies demonstrating that microspheres having an average diameter of about 4.2 μm suppress sebaceous gland in the hamster flank organ model in a dose dependent manner. The dose of tazarotene is indicated as ng at the bottom. The level of sebaceous gland at each point is expressed as percent of control side. As an example, a 60% control side means 40% inhibition. The clinical strength of 0.1 wt % (10 μl or 10,000 ng) Tazorac® (tazarotene) gel did not significantly reduce sebaceous gland size. A biphasic dose curve is observed with tazarotene microsphere. Significance levels: *p<0.05, **p<0.01, ***p<0.001.

FIG. 3 shows the detection of fluorescent tazarotene microsphere in a hair follicle on pig ear skin. The arrow point to a signal detected at depth greater than 500 μm. Skin surface is indicated by a yellow line. The strong signals are concentrated at depth of 100-200 μm. Sebaceous glands (not found in this follicle) are normally at 400-500 μm. Fresh pig ear was treated with tazarotene microsphere containing 0.1% tazarotene in PBS (phosphate buffered saline) by rubbing with a glass rod for 2 min. Vertical slices (100 nm thick) of the treated area was obtained by cryosectioning and they were examined under a microscope with 340 nm UV excitation.

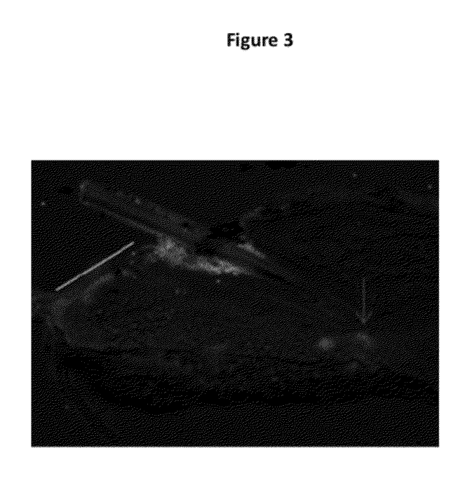

### DETAILED DESCRIPTION

The compositions of the invention comprise particles having an average diameter between about 0.1 μm and about 10 μm, and wherein the particles comprise a) a biodegradable polymer, and b) retinoid compounds as defined below.

### Retinoid Compounds of the Invention

In one embodiment, the compositions of the invention comprise compounds having the following formula (Formula I):

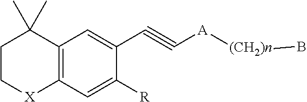

wherein

X is S, O, or NR= where R= is hydrogen or lower alkyl;

R is hydrogen or lower alkyl;

A is pyridinyl, thienyl, furyl, pyridazinyl, pyrimidinyl or pyrazinyl;

n is 0-2;

B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4.

Methods for synthesizing compounds of Formula I are described in U.S. Pat. No. 5,089,509 and U.S. Patent Application Publication No. 2011/0076318, the contents of both of which are incorporated herein by reference.

In one embodiment, compounds of Formula I include compounds where the ethynyl group and the B group are attached to the 2 and 5 positions respectively of a pyridine ring (the 6 and 3 positions in the nicotinic acid nomenclature being equivalent to the ⅖ designation in the pyridine nomenclature) or the 5 and 2 positions respectively of a thiophene group respectively; n is 0; and B is —COOH, an alkali metal salt or organic amine salt, or a lower alkyl ester, or —CH₂OH and the lower alkyl esters and ethers thereof, or —CHO and acetal derivatives thereof.

In another embodiment, compounds of Formula I include the following:

ethyl 6-(2-(4,4-dimethylthiochroman-6-yl)ethynyl)-nicotinate;

6-(2-(4,4-dimethylthiochroman-6-yl)ethynyl)nicotinic acid;

6-(2-(4,4-dimethylchroman-6-yl)ethynyl)nicotinic acid;

ethyl 6-(2-(4,4-dimethylchroman-6-yl)ethynyl) nicotinate;

ethyl 6-(2-(4,4,7-trimethylthiochroman-6-yl)ethynyl)-nicotinate;

ethyl 6-(2-(4,4-dimethyl-1,2,3,4-tetrahydroquinolin-6-yl)ethynyl)nicotinate;

ethyl 5-(2-(4,4-dimethylthiochroman-6-yl)ethynyl)-thiophene-2-carboxylate;

6-(2-(4,4-dimethylthiochroman-6-yl)ethynyl)-3-pyridylmethanol; and

2-(2-(4,4-dimethylthiochroman-6-yl)ethynyl)-5-pyridinecarboxaldehyde.

In one embodiment, the composition of the invention comprises tazarotene, tazarotenic acid, or mixtures thereof. Tazarotene, a compound of Formula I, has the following structure:

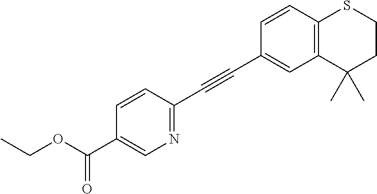

Its chemical name is ethyl 6-[2-(4,4-dimethylthiochroman-6-yl)ethynyl]nicotinate. Tazarotenic acid, another compound of Formula I, has the following structure:

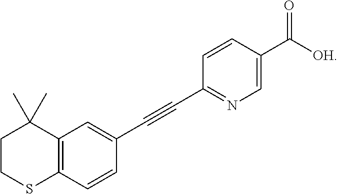

Its chemical name is 6-(2-(4,4-dimethylthiochroman-6-yl)ethynyl)nicotinic acid. Both tazarotene and tazarotenic acid may be synthesized according to the methods described in U.S. Pat. No. 5,089,509.

In another embodiment, compounds of Formula I include the following:

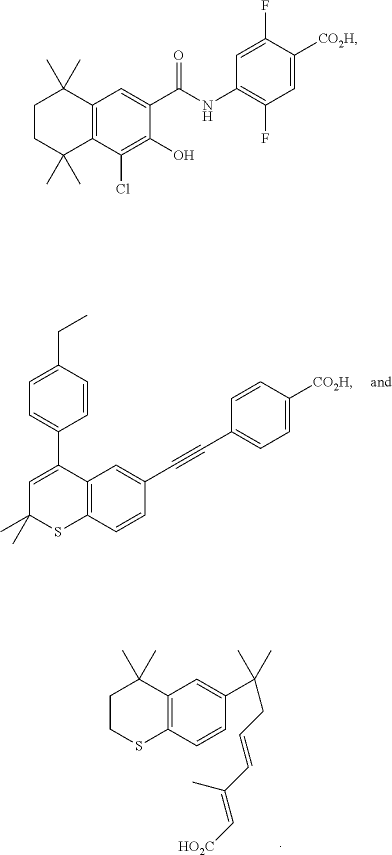

These compounds may also be synthesized according to the methods described in U.S. Pat. No. 5,089,509.

In another embodiment, the compositions of the invention comprise compounds having the following formula (Formula II):

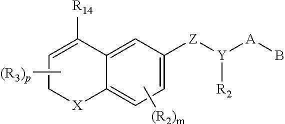

wherein X is S, O, NR′ where R′ is H or alkyl of 1 to 6 carbons, or

X is [C(R₁)₂]ₙ where R₁ is independently H or alkyl of 1 to 6 carbons, and n is an integer between, and including, 0 and 2, and;

R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;

R₃ is hydrogen, lower alkyl of 1 to 6 carbons or F, and;

m is an integer having the value of 0-3, and;

p is an integer having the value of 0-3, and;

Z is —C≡C—, —N═N—, —N═CR₁—, —CR₁═N, —(CR₁═CR₁)_{n′}— where n′ is an integer having the value 0-5, —CO—NR₁—, —CS—NR₁—,—NR₁—CO, —NR₁—CS, —COO—, —OCO—; —CSO—; —OCS—;

Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′}— group and B;

A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;

B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and

R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and

R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons.

Methods for synthesizing compounds of Formula II are described in U.S. Pat. No. 5,776,699, the contents of which are incorporated herein by reference, and in U.S. Patent Application Publication No. 2011/0076318.

As used throughout this application, the term “alkyl refers to any and all groups which are known as normal alkyl, branched-chain alkyl, and cycloalkyl. The term “alkenyl” refers to normal alkenyl, branch chain alkenyl, and cycloalkenyl groups having one or more sites of unsaturation. Similarly, the term “alkynyl” refers to normal alkynyl, and branch chain alkynyl groups having one or more triple bonds.

“Lower alkyl” means the above-defined broad definition of alkyl groups having 1 to 6 carbons in case of normal lower alkyl, and as applicable 3 to 6 carbons for lower branch chained and cycloalkyl groups. “Lower alkenyl” is defined similarly having 2 to 6 carbons for normal lower alkenyl groups, and 3 to 6 carbons for branch chained and cyclo- lower alkenyl groups. “Lower alkynyl” is also defined similarly, having 2 to 6 carbons for normal lower alkynyl groups, and 4 to 6 carbons for branch chained lower alkynyl groups.

The term “ester” refers to any compound falling within the definition of that term as classically used in organic chemistry. It includes organic and inorganic esters. Where B (of Formula I) is -COON, this term covers the products derived from treatment of this function with alcohols or thiols preferably with aliphatic alcohols having 1-6 carbons. Where the ester is derived from compounds where B is —CH₂OH, this term covers compounds derived from organic acids capable of forming esters including phosphorous based and sulfur based acids, or compounds of the formula —CH₂OCORH₁₁ where R₁₁ is any substituted or unsubstituted aliphatic, aromatic, heteroaromatic or aliphatic aromatic group, preferably with 1-6 carbons in the aliphatic portions.

Unless stated otherwise in this application, esters are derived from the saturated aliphatic alcohols or acids of ten or fewer carbon atoms or the cyclic or saturated aliphatic cyclic alcohols and acids of 5 to 10 carbon atoms. Examples include aliphatic esters derived from lower alkyl acids and alcohols, and phenyl or lower alkyl phenyl esters.

The term “amide” has the meaning classically accorded that term in organic chemistry. In this instance it includes the unsubstituted amides and all aliphatic and aromatic mono- and di- substituted amides. Examples include the mono- and di-substituted amides derived from the saturated aliphatic radicals of ten or fewer carbon atoms or the cyclic or saturated aliphatic-cyclic radicals of 5 to 10 carbon atoms. In one embodiment, the amides are derived from substituted and unsubstituted lower alkyl amines. In another embodiment, the amides are mono- and disubstituted amides derived from the substituted and unsubstituted phenyl or lower alkylphenyl amines. One may also use unsubtituted amides.

“Acetals” and “ketals” include the radicals of the formula-CK where K is (—OR)₂. Here, R is lower alkyl. Also, K may be —OR₇O— where R₇ is lower alkyl of 2-5 carbon atoms, straight chain or branched.

The compounds of the invention may be used as their pharmaceutically acceptable salts. Pharmaceutically acceptable acid addition salts of the compounds of the systems are those formed from acids which form non-toxic addition salts containing pharmaceutically acceptable anions, such as the hydrochloride, hydrobromide, hydroiodide, sulfate, or bisulfate, phosphate or acid phosphate, acetate, maleate, fumarate, oxalate, lactate, tartrate, citrate, gluconate, saccharate and p-toluene sulphonate salts.

A pharmaceutically acceptable salt may be prepared for any compounds in this invention having a functionality capable of forming a salt, for example an acid functionality. A pharmaceutically acceptable salt is any salt which retains the activity of the parent compound and does not impart any deleterious or untoward effect on the subject to which it is administered and in the context in which it is administered.

Pharmaceutically acceptable salts may be derived from organic or inorganic bases. The salt may be a mono or polyvalent ion. Of particular interest are the inorganic ions, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Where there is a nitrogen sufficiently basic as to be capable of forming acid addition salts, such may be formed with any inorganic or organic acids or alkylating agent such as methyl iodide. Preferred salts are those formed with inorganic acids such as hydrochloric acid, sulfuric acid or phosphoric acid. Any of a number of simple organic acids such as mono-, di- or tri- acid may also be used.

Some of the compounds of the present invention may have trans and cis (E and Z) isomers. In addition, the compounds of the present invention may contain one or more chiral centers and therefore may exist in enantiomeric and diastereomeric forms. The scope of the present invention is intended to cover all such isomers per se, as well as mixtures of cis and trans isomers, mixtures of diastereomers and racemic mixtures of enantiomers (optical isomers) as well. In the present application when no specific mention is made of the configuration (cis, trans or R or S) of a compound (or of an asymmetric carbon) then a mixture of such isomers, or either one of the isomers is intended.

### Biodegradable Particles

The inventors have discovered that one can deliver the retinoids of the invention to the skin using particles of biodegradable polymer, wherein the particles have an average diameter no less than about 0.1 μm and no greater than about 10 μm

In one embodiment, the particle is shaped like a sphere. The inventors refer to such particles as “microspheres,” even though they may have an average diameter in the nanometer range (that is, about 100 nm to about 999 nm). The microspheres of the invention have a maximum average diameter of about 10 μm.

As used here, the term “about,” when used in connection with a value, means that the value may not differ by more than 5%. Hence, “about 10 μm” includes all values within the range of 9.5 μm to 10.5 μm.

In one embodiment, the microspheres of the invention have a maximum average diameter of about 10 μm. In another embodiment, the microspheres of the invention have a maximum average diameter of about 9 μm. In another embodiment, the microspheres of the invention have a maximum average diameter of about 8 μm. In another embodiment, the microspheres of the invention have a maximum average diameter of about 7 μm. In another embodiment, the microspheres of the invention have a maximum average diameter of about 6 μm. In another embodiment, the microspheres of the invention have a maximum average diameter of about 5 μm. In another embodiment, the microspheres of the invention have a maximum average diameter of about 4 μm. In another embodiment, the microspheres of the invention have a maximum average diameter of about 3 μm. In another embodiment, the microspheres of the invention have a maximum average diameter of about 2 μm. In another embodiment, the microspheres of the invention have a maximum average diameter of about 1 μm.

In another embodiment, the microspheres of the invention have a maximum average diameter less than about 1 μm. In another embodiment, the microspheres of the invention have a maximum average diameter of about 0.9 μm. In another embodiment, the microspheres of the invention have a maximum average diameter of about 0.8 μm. In another embodiment, the microspheres of the invention have a maximum average diameter of about 0.7 μm. In another embodiment, the microspheres of the invention have a maximum average diameter of about 0.6 μm. In another embodiment, the microspheres of the invention have a maximum average diameter of about 0.5 μm. In another embodiment, the microspheres of the invention have a maximum average diameter of about 0.4 μm. In another embodiment, the microspheres of the invention have a maximum average diameter of about 0.3 μm. In another embodiment, the microspheres of the invention have a maximum average diameter of about 0.2 μm. In another embodiment, the microspheres of the invention have a maximum average diameter of about 0.1 μm.

In one embodiment, the particle is shaped like a cylindrical rod. The inventors refer to such particles as “microcylinders,” even though they may have an average diameter in the nanometer range (that is, about 100 nm to about 999 nm). The microcylinders of the invention have a maximum average diameter and maximum average length such that no one such dimension is greater than about 10 μm. In other embodiments, the particles of the invention are of different geometry, such as fibers or circular discs; any geometry falls within the scope of the invention, as long as the average of any single dimension of the particle exceeds about 10 μm.

In one embodiment, the microcylinders of the invention have a maximum average diameter of about 10 μm. In another embodiment, the microcylinders of the invention have a maximum average diameter of about 9 μm. In another embodiment, the microcylinders of the invention have a maximum average diameter of about 8 μm. In another embodiment, the microcylinders of the invention have a maximum average diameter of about 7 μm. In another embodiment, the microcylinders of the invention have a maximum average diameter of about 6 μm. In another embodiment, the microcylinders of the invention have a maximum average diameter of about 5 μm. In another embodiment, the microcylinders of the invention have a maximum average diameter of about 4 μm. In another embodiment, the microcylinders of the invention have a maximum average diameter of about 3 μm. In another embodiment, the microcylinders of the invention have a maximum average diameter of about 2 μm. In another embodiment, the microcylinders of the invention have a maximum average diameter of about 1 μm.

In another embodiment, the microcylinders of the invention have a maximum average diameter less than about 1 μm. In another embodiment, the microcylinders of the invention have a maximum average diameter of about 0.9 μm. In another embodiment, the microcylinders of the invention have a maximum average diameter of about 0.8 μm. In another embodiment, the microcylinders of the invention have a maximum average diameter of about 0.7 μm. In another embodiment, the microcylinders of the invention have a maximum average diameter of about 0.6 μm. In another embodiment, the microcylinders of the invention have a maximum average diameter of about 0.5 μm. In another embodiment, the microcylinders of the invention have a maximum average diameter of about 0.4 μm. In another embodiment, the microcylinders of the invention have a maximum average diameter of about 0.3 μm. In another embodiment, the microcylinders of the invention have a maximum average diameter of about 0.2 μm. In another embodiment, the microcylinders of the invention have a maximum average diameter of about 0.1 μm.

In one embodiment, the microcylinders have a maximum average length of about 10 μm, about 9 μm, about 8 μm, about 7 μm, about 6 μm, about 5 μm, about 4 μm, about 3 μm, about 2 μm, about 1 μm, about 0.9 μm, about 0.8 μm, about 0.7 μm, about 0.6 μm, about 0.5 μm, about 0.4 μm, about 0.3 μm, or about 0.2 μm.

The size and geometry of the particle can also be used to control the rate of release, period of treatment, and drug concentration. Larger particles will deliver a proportionately larger dose, but, depending on the surface to mass ratio, may have a slower release rate.

The retinoid of the invention may be in a particulate or powder form. In one embodiment, the retinoid itself consists of particles having the dimensions described above.

In another embodiment, the retinoids are combined with a biodegradable polymer. In one embodiment, the retinoid is from about 10% to about 90% by weight of the composition. In another embodiment, the retinoid is from about 20% to about 80% by weight of the composition. In another embodiment, the retinoid is from about 30% to about 70% by weight of the composition. In another embodiment, the retinoid is from about 40% to about 60% by weight of the composition. In one embodiment, the retinoid comprises about about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the composition.

Suitable polymeric materials for use in the compositions of the invention include those materials which are biocompatible with the skin so as to cause no substantial irritation or other side effects. In one embodiment, such materials are at least partially biodegradable. In another embodiment, such materials are completely biodegradable.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, CRC Critical Reviews in Therapeutic Drug Carrier Systems, Vol. 1, CRC Press, Boca Raton, Fla. 1987, pp 39-90 (Biodegradable Polymers in Controlled Drug Delivery), the contents of which are incorporated herein by reference, which describes encapsulation for controlled drug delivery, may find use in the present compositions.

Of additional interest are polymers of hydroxyaliphatic carboxylic acids, either homopolymers or copolymers, and polysaccharides, lipid nanoparticle, and mesoporous silica nanoparticle. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

Some preferred characteristics of the polymers or polymeric materials for use in the present invention may include biocompatibility, compatibility with the therapeutic compound, ease of use of the polymer in making the compositions of the present invention, a half-life in the physiological environment of at least about 6 hours, preferably greater than about one day, not significantly increasing the viscosity of the vitreous, and water insolubility.

The biodegradable polymeric materials which are included to form the particles are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the system is the relative average molecular weight of the polymeric composition employed in the system. Different molecular weights of the same or different polymeric compositions may be included in the system to modulate the release profile. In certain systems, the relative average molecular weight of the polymer will range from about 9 to about 64 kD, from about 10 to about 54 kD, or from about 12 to about 45 kD.

In some compositions, copolymers of glycolic acid and lactic acid (poly(lactic-co-glycolic acid)) are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the drug delivery system, where a more flexible system is desirable for larger geometries. The proportion of polylactic acid in the polylactic acid polyglycolic acid (PLGA) copolymer can be 0-100%; in other embodiments, the proportion of polylactic acid can be from about 10% to about 90%, from about 20% to about 80%, from about 30% to about 70%,or from about 40% to about 60%. In one embodiment, the proportion of polylactic acid may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the composition.

The biodegradable polymer of the composition of the invention may comprise a mixture of two or more biodegradable polymers. For example, the composition may comprise a mixture of a first biodegradable polymer and a different second biodegradable polymer. One or more of the biodegradable polymers may have terminal acid groups.

Release of a drug from an erodible polymer is the consequence of several mechanisms or combinations of mechanisms. Some of these mechanisms include desorption from the systems surface, dissolution, diffusion through porous channels of the hydrated polymer and erosion. Erosion can be bulk or surface or a combination of both.

One example of a composition of the invention comprises tazarotene, tazarotenic acid, or a combination thereof with a biodegradable polymer matrix that comprises a (lactide-co-glycolide) or a poly (D,L-lactide-co-glycolide). The composition system may have an amount of the retinoid compound from about 40% to about 70% by weight of the system.

The release of the retinoid from the composition may include an initial burst of release followed by a gradual increase in the amount of the retinoid released, or the release may include an initial delay in release of the retinoid followed by an increase in release. When the biodegradable polymer is substantially completely degraded, the percent of the retinoid that has been released is about one hundred percent.

It may be desirable to provide a relatively constant rate of release of the retinoid from the particles. However, the release rate may change to either increase or decrease depending on the formulation of the particle. In addition, the release profile of the retinoid may include one or more linear portions and/or one or more non-linear portions. In one embodiment, the release rate is greater than zero once the system has begun to degrade or erode.

The particles of the invention may be monolithic, that is, having the active agent or agents homogenously distributed through the polymer, or encapsulated, where a reservoir of active agent is encapsulated by the polymer. Due to ease of manufacture, monolithic systems are usually preferred over encapsulated forms. However, the greater control afforded by the encapsulated, reservoir-type implants may be of benefit in some circumstances, where the therapeutic level of the drug falls within a narrow window. In addition, the therapeutic compound, including the retinoid compound, may be distributed in a non-homogenous pattern in the polymer. For example, a particle may include a portion that has a greater concentration of the retinoid compound relative to a second portion of the implant.

Thus, particles can be prepared where the center may be of one material and the surface may have one or more layers of the same or a different material, where the layers may be cross-linked, or of a different molecular weight, different density or porosity, or the like. For example, where it is desirable to quickly release an initial bolus of drug, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve.

The proportions of retinoid compound, polymer, and any other modifiers may be empirically determined by formulating several drug delivery systems with varying proportions. A USP approved method for dissolution or release test can be used to measure the rate of release (USP 23; NF 18 (1995) pp. 1790-1798). For example, using the infinite sink method, a weighed sample of the implant is added to a measured volume of a solution containing 0.9% NaCl in water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to the retinoid compound and polymer, the particles disclosed herein may include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total drug delivery system. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from about 0.001% to about 5% by weight; in another embodiment, they may be present in amounts from about 0.01% to about 2% by weight.

In addition, the particles may include a solubility enhancing compound provided in an amount effective to enhance the solubility of the retinoid compound relative to substantially identical systems without the solubility enhancing compound. For example, an implant may include a δ-cyclodextrin, which is effective in enhancing the solubility of the retinoid compound. The δ-cyclodextrin may be provided in an amount from about 0.5% (w/w) to about 25% (w/w) of the particle. In other embodiments, the δ-cyclodextrin is provided in an amount from about 5% (w/w) to about 15% (w/w) of the particle.

Additionally, release modulators such as those described in U. S. Pat. No. 5,869,079, the contents of which are incorporated herein by reference, may be included in the particles. The amount of release modulator employed will be dependent on the desired release profile, the activity of the modulator, and on the release profile of the retiniod in the absence of modulator. Electrolytes such as sodium chloride and potassium chloride may also be included in the implant. Where the buffering agent or enhancer is hydrophilic, it may also act as a release accelerator. Hydrophilic additives act to increase the release rates through faster dissolution of the material surrounding the drug particles, which increases the surface area of the drug exposed, thereby increasing the rate of drug bioerosion. Similarly, a hydrophobic buffering agent or enhancer dissolve more slowly, slowing the exposure of drug particles, and thereby slowing the rate of drug bioerosion.

Various techniques may be employed to produce the particles described herein. In one embodiment, particles are produced using a solvent evaporation process. Such a process may include steps of liquid sieving, freeze drying, and sterilizing the various composition compounds. In one embodiment, a retinoid compound and a polymer are combined with methylene chloride to form a first composition, and water and polyvinyl alcohol are combined to form a second composition. The first and second compositions are combined to form an emulsion. The emulsion is rinsed and/or centrifuged, and the resulting product dried. In a further embodiment, the emulsion undergoes an evaporation process to remove methylene chloride from the emulsion. For example, the emulsion can be evaporated for about 2 days or more. In this embodiment, the method comprises sieving retinoid-containing microspheres in a liquid phase, as compared to a method which comprises sieving retinoid-containing microparticles in a dry phase. This method can also comprise a step of freeze drying the sieved microparticles, and a step of packaging the freeze dried microparticles.

In another embodiment, a method of producing retinoid-containing microspheres comprises one or more of the following steps. In certain embodiments, the method comprises each of the following steps. A polymeric material, such as PLGA, is dissolved in a solvent, such as methylene chloride. The dissolving of the PLGA can occur with stirring the mixture until the PLGA is completely dissolved. A predetermined amount of a retinoid compound, such as tazarotene, is added to the dissolved PLGA composition. The resulting composition can be understood to be a first composition in reference to this method. A second different composition is produced by combining heated water, for example water having a temperature of about 80 degrees C., with polyvinylic alcohol (PVA). The PVA can be combined with the heated water by stirring the water at a rate effective in maintaining PVA in suspension without substantial bubble formation. The second composition may then be cooled to a desired temperature, such as room temperature.

An emulsion can be produced by combining the first composition and the second composition described in the preceding paragraph. For example, the second composition (i.e., the PVA solution) can be vigorously stirred while avoiding bubble formation. While stirring the second composition, the first composition is added to form an emulsion. As the mixture emulsifies, the stirring speed may be increased to keep the surface of the emulsion moving. Foam or bubble formation is minimized during these steps. In this method, the emulsion is stirred for at least two days (e.g., for about 48 hours or more). As the emulsion is stirred for about 24 hours, the emulsion begins to liquefy. To reduce the possibility of foaming, the stirring speed can be decreased as the emulsion liquefies. After about 48 hours, methylene chloride is substantially or completely evaporated. The method can include a step of determining the amount of methylene chloride in the evaporated material.

After the evaporation of the methylene chloride, the microparticle-containing composition is rinsed and sieved. For example, the microparticle-containing composition is combined with a liquid and centrifuged. The supernatant is removed and the pellet can be resuspended by sonication or other suitable method for additional centrifugation steps. After the microsphere suspension has been centrifuged, water can be added to rinse the microspheres, and the resulting supernatant can be removed by vacuum extraction. In preferred methods, at least three water rinsing steps are desirable. The rinsed pellets are then sieved through a plurality of filters. For example, the pellets can be passed through two superimposed filters having a pore size of about 125 μm and about 45 μm, respectively. The filters can be rinsed with water and the solution can be retrieved in the filter bottom.

The retrieved solution can then be combined with an additional amount of water and rinsed two or more times using a centrifuge. The rinsed pellet can then be placed in the filter bottom and covered with a filter to reduce loss of the microsphere material during a lyophilization procees. The material is then frozen. For example, the material is frozen at fifty degrees C. and freeze dried for at least twelve hours. After freeze drying, the microspheres can be stored in a package, and/or may be sterilized by a sterilization device, such as a source of gamma radiation.

Additional examples of methods for producing retinoid-containing particles are described in U.S. Patent Application Publication No. 2011/0076318. Additional examples of producing particles of biodegradable polymer may be found in U.S. Patent Application Publication No. 2005/0003007 and No. 2008/0182909, the contents of both of which are incorporated herein by reference.

Conditions Associated with Excess Sebum Production

In one embodiment, the compositions of the invention may be used to treat conditions associated with excess sebum production. Such conditions include, for example, acne vulgaris, seborrhoeic dermatitis, keratosis pilaris,

In another embodiment, the compositions of the invention may be used to treat those conditions in which it would be beneficial to suppress the function of the sebaceous gland. Such conditions include, for example, sebaceous cyst, sebaceous hyperplasia, sebaceous adenoma, and sebaceous gland carcinoma.

### EXAMPLES

The invention is illustrated further with the following examples.

### Tazarotene Microspheres

The inventors prepared a composition comprising PLGA microspheres having an average diameter of about 4.2 μm and containing 0.03% tazarotene.

### Animals and Treatment Procedure

The inventors used male hamsters weighing about 110-120 g. The animals arrived at least 7 days before the study and were single-housed. Animals are randomized by weight. The inventors shave the right side flank to expose the flank organ, removing as much hair as possible, and wiped the animals clean with a cotton swab soaked with 70% ethanol.

The inventors applied the 0.03% tazarotene 4.2 μm microspheres with a pipette and carefully spread it over the flank organ. Each time before applying the drug, the inventors wiped clean the flank organ area with a cotton swab soaked with 70% ethanol. The inventors treated animals in this manner 5 days/week for 26 days. If hair grew back on the flank organ the inventors shaved it.

### Tissue Processing and Analysis

The inventors sacrificed animals with CO₂ and then shaved, cleaned, and excised the flank organ. They attached the organ to a paper card, put it into a thick cassette, overlaying with a piece of sponge, and closed it. They air dried the cassette for a few minutes before putting it into 10% formalin (buffered) for fixation.

The inventors cut at the middle of the organ to make 15-20 μm slices, put the slices onto glass slides, and then stained with hematoxylin and eosin. The inventors scanned the slides with NanoZoomer® to obtain clear pictures and measured the sebaceous gland areas with the software accompanying the NanoZoomer®.

The inventors used a pair wise t-TEST to compare treated sides against untreated control sides, and used a one-way ANOVA analysis to compare the drug treated group against the vehicle treated group.

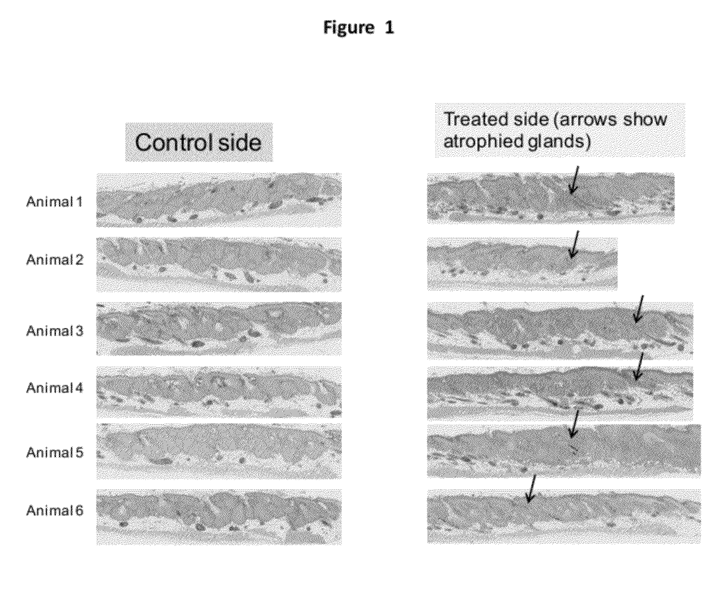

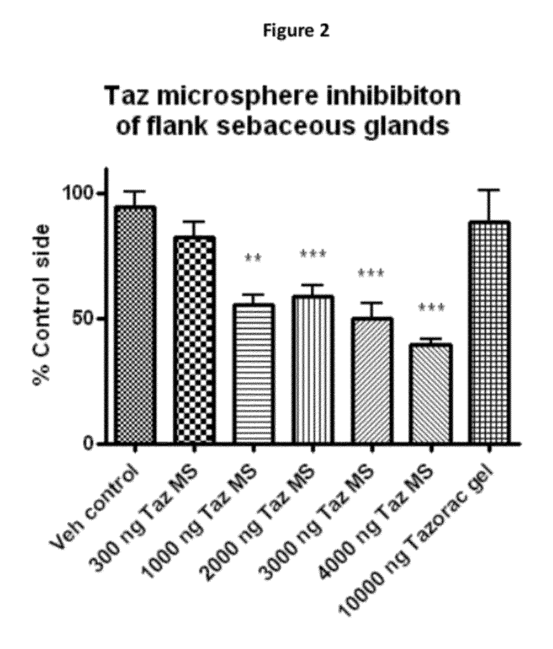

The results of the experiment are shown in FIGS. 1 and 2.

Each and every reference is incorporated herein by reference in its entirety for all purposes.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A dermal topical composition comprising particles, wherein the particles comprise
a) a biodegradable polymer, and
b) a compound of the formula: 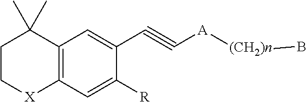
wherein:
X is S, O, or —N(R¹)— where R¹ is hydrogen or lower alkyl;
R is hydrogen or lower alkyl;
A is pyridinyl, thienyl, furyl, pyridazinyl, pyrimidinyl or pyrazinyl;
n is 0-2;
B is selected from the group consisting of: H, —COOH or a pharmaceutically acceptable salt, ester or amide of said —COOH group, —CH₂OH or an ether or ester derivative of said —CH₂OH group , —CHO or an acetal derivative of said —CHO group, and —COR² or a ketal derivative of said —COR² group, wherein R² is —(CH₂)ₘCH₃ wherein m is 0-4; and
wherein the particles have an average diameter between about 0.1 μm and about 10 μm.

2. The composition of claim 1, wherein the particles have an average diameter no greater than about 5 μm.

3. The composition of claim 1, wherein the particles have an average diameter no greater than about 4 μm.

4. The composition of claim 1, wherein the particles have an average diameter no greater than about 1 μm.

5. The composition of claim 1, wherein the biodegradable polymer is selected from the group consisting of poly hydroxyaliphatic carboxylic acids, polyesters, polysaccharides, and combinations thereof.

6. The composition of claim 1, wherein the biodegradable polymer is poly(lactic-co-glycolic acid) (PLGA).

7. The composition of claim 1, wherein the particles are spheres.

8. The composition of claim 1, wherein the particles are cylinders.

9. The composition of claim 1, wherein the compound is tazarotene.

10. The composition of claim 1, wherein the compound is tazarotenic acid or a pharmaceutically acceptable salt, ester or amide thereof.

11. A method for treating a condition associated with excess sebum production, the method comprising topically applying to the skin of a patient in need of such treatment the composition of claim 1.

12. The method of claim 11, wherein the condition is selected from the group consisting of: acne vulgaris, seborrhoeic dermatitis, and keratosis pilaris.

13. The method of claim 11, wherein the composition provides for an extended release of the compound

14. A dermal topical composition comprising particles, wherein the particle comprise
a) a biodegradable polymer, and
b) a compound of the formula: 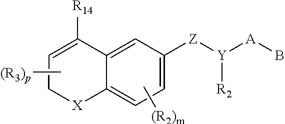
or a pharmaceutically acceptable salt thereof, wherein X is S, O, NR′ where R′ is H or alkyl of 1 to 6 carbons, or
X is [C(R₁)₂]ₙ where R₁ is independently H or alkyl of 1 to 6 carbons, and n is an integer between, and including, 0 and 2, and;
R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;
R₃ is hydrogen, lower alkyl of 1 to 6 carbons or F, and;
m is an integer having the value of 0-3, and;
p is an integer having the value of 0-3, and;
Z is —C≡C—, —N═N—, —N═CR₁—, —CR₁═N, —(CR₁═CR₁)_{n′}— where n′ is an integer having the value 0-5, —CO—NR₁—, —CS—NR₁—, —NR₁—CO, —NR₁—CS, —COO—, —OCO—; —CSO—; —OCS—;
Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′}— group and B;
A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and
R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and
R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons;
and wherein the particles have an average diameter between 0.1 μm and 10 μm.

15. The composition of claim 14, wherein the particles have an average diameter no greater than about 10 μm.

16. The composition of claim 14, wherein the particles have an average diameter no greater than about 5 μm.

17. The composition of claim 14, wherein the particles have an average diameter no greater than about 1 μm.

18. The composition of claim 14, wherein the biodegradable polymer is PLGA.

19. The composition of claim 14, wherein the particles are spheres.

20. The composition of claim 14, wherein the particles are cylinders.

21. A method for treating a condition associated with excess sebum production, the method comprising topically applying to the skin of a patient in need of such treatment the composition of claim 14.

22. The method of claim 21, wherein the condition is selected from acne vulgaris, seborrhoeic dermatitis, and keratosis pilaris.

23. The method of claim 14, wherein the composition provides for an extended release of the compound.
